(19) United States
(12) Patent Application Publication
(10) Pub. No.: US 2025/0032387 A1
(43) Pub. Date: Jan. 30, 2025

(54) A PERSONAL CARE OR PHARMACEUTICAL COMPOSITION

(71) Applicant: Conopco, Inc., d/b/a UNILEVER, Englewood Cliffs, NJ (US)

(72) Inventors: Xuelan GU, Shanghai (CN); Tingyan MI, Shanghai (CN)

(73) Assignee: Conopco, Inc., d/b/a UNILEVER, Englewood Cliffs, NJ (US)

(21) Appl. No.: 18/716,953

(22) PCT Filed: Dec. 12, 2022
(86) PCT No.: PCT/EP2022/085284

§ 371 Date: Jun. 6, 2024

(30) Foreign Application Priority Data
Dec. 31, 2021 (WO) PCT/CN2021/143517
Jan. 27, 2022 (EP) 22153549.5

(51) Int. Cl. A61K 8/67; A61K 8/44; A61K 31/198; A61K 31/455; A61P 17/02; A61Q 19/00

(52) U.S. Cl. CPC A61K 8/675; A61K 8/44; A61K 31/198; A61K 31/455; A61P 17/02; A61Q 19/00

(57) ABSTRACT

Disclosed is a personal care or pharmaceutical composition comprising vitamin B3 compound and carboxymethyl cysteine compound, wherein the weight ratio of the vitamin B3 compound to the carboxymethyl cysteine compound is at least 6:1.

### FIELD OF THE INVENTION

The present invention relates to a personal care or pharmaceutical composition comprising vitamin B3 compound and carboxymethyl cysteine compound for skin wound healing and/or skin regeneration.

### BACKGROUND OF THE INVENTION

The skin is a primary barrier of the human body and protects the organs in the body from stimulation caused by external environments such as ultraviolet rays, pollutants, temperature and etc.

Wound healing is the restoration in injured skin tissue. Delayed or impaired wound healing may cause cosmetic problems or even severe medical conditions, like ulcers. Therefore, treatments to improve wound healing is important in both cosmetic and pharmaceutical applications. During wound healing, various reactions are involved. Migration of keratinocytes is one of the particularly important processes for wound healing or skin regeneration.

Therefore, the present inventors have recognized that there is a need to develop solution to improve migration rate of keratinocytes for wound healing and/or skin regeneration. It was surprisingly found that by combining and vitamin B3 compound in a specific weight ratio, the migration rate of keratinocytes was significantly enhanced.

### SUMMARY OF THE INVENTION

In a first aspect, the present invention is directed to a personal care or pharmaceutical composition comprising vitamin B3 compound and carboxymethyl cysteine compound, wherein the weight ratio of the vitamin B3 compound to the carboxymethyl cysteine compound is at least 6:1.

In a second aspect, the present invention is directed to a method of healing and/or alleviating a skin wound; and/or regenerating skin comprising a step of topically applying to the skin the composition of the present invention.

In a third aspect, the present invention is directed to use of the composition of the present invention for healing and/or alleviating a skin wound; and/or regenerating skin.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

Vitamin B3 compound as used herein also comprises niacin, niacinamide, nicotinyl alcohol, derivatives thereof, salts of any of the foregoing, as well as mixtures thereof. Derivatives typically comprise nicotinic acid esters, nicotinyl alcohol esters of carboxylic acids, niacinamide N-oxide, nicotinyl amino acids and/or nicotinic acid n-oxide. Preferably, the vitamin B3 compound comprises niacin, niacinamide, or a mixture thereof. More preferably, the vitamin B3 compound is selected from niacin, niacinamide or a mixture thereof. Even more preferably the vitamin B3 compound comprises niacinamide and most preferably the vitamin B3 compound is niacinamide.

Preferably, the vitamin B3 compound is present in an amount of at least 0.00001%, more preferably at least 0.0001%, even more preferably at least 0.001%, still even more preferably at least 0.01% and most preferably at least 0.03% by weight of the composition. Typically, the vitamin B3 compound is present in an amount of no more than 10%, preferably no more than 5%, more preferably no more than 2%, even more preferably no more than 0.5% by weight of the composition. Preferably, the niacinamide is present in an amount of at least 0.00001%, more preferably at least 0.0001%, even more preferably at least 0.001%, still even more preferably at least 0.01% and most preferably at least 0.03% by weight of the composition. Typically, the niacinamide is present in an amount of no more than 10%, preferably no more than 5%, more preferably no more than 2%, even more preferably no more than 0.5% by weight of the composition.

The carboxymethyl cysteine compound refers to compound selected from carboxymethyl cysteine, salt of carboxymethyl cysteine, ester of carboxymethyl cysteine, amide of carboxymethyl cysteine or a mixture thereof. Preferably, the carboxymethyl cysteine compound comprises carboxymethyl cysteine, ester of carboxymethyl cysteine, and/or salt of carboxymethyl cysteine. More preferably, the carboxymethyl cysteine compound comprises carboxymethyl cysteine, and/or salt of carboxymethyl cysteine. Even more preferably, carboxymethyl cysteine compound comprises salt of carboxymethyl cysteine. Still even more preferably the carboxymethyl cysteine compound comprises lysine carboxymethyl cysteinate and most preferably, the carboxymethyl cysteine compound is lysine carboxymethyl cysteinate.

Preferably, the carboxymethyl cysteine compound is present in amount of at least 0.00001%, more preferably at least 0.0001%, even more preferably at least 0.001%, still even more preferably at least 0.01%, and most preferably at least 0.1% by weight of the composition. Preferably, the carboxymethyl cysteine compound is present in amount of no greater than 10%, more preferably no greater than 5%, even more preferably no greater than 3%, still even more preferably no greater than 1%, and most preferably no greater than 0.5% by weight of the composition.

Preferably, the lysine carboxymethyl cysteinate is present in amount of no greater than 10%, more preferably no greater than 5%, even more preferably no greater than 3%, still even more preferably no greater than 1%, and most preferably no greater than 0.5% by weight of the composition. Preferably, the lysine carboxymethyl cysteinate is present in amount of at least 0.00001%, more preferably at least 0.0001%, even more preferably at least 0.001%, still even more preferably at least 0.01%, and most preferably at least 0.1% by weight of the composition.

Preferably, the weight ratio of the vitamin B3 compound to the carboxymethyl cysteine compound is 6:1 to 10000:1, more preferably 6:1 to 1000:1, even more preferably 6:1 to 100:1, still even more preferably 7:1 to 50:1 and most preferably 8:1 to 20:1. Preferably, the weight ratio of the vitamin B3 compound to the lysine carboxymethyl cysteinate is 6:1 to 10000:1, more preferably 6:1 to 1000:1, even more preferably 6:1 to 100:1, still even more preferably 7:1 to 50:1 and most preferably 8:1 to 20:1.

The composition may optionally comprise whitening pigment. Whitening pigments are typically particles of high refractive index materials. For example, the whitening pigment may have a refractive index of greater than 1.3, more preferably greater than 1.8 and most preferably from 2.0 to 2.7. Examples of such whitening pigment are those comprising bismuth oxy-chloride, boron nitride, barium sulfate, mica, silica, titanium dioxide, zirconium oxide, aluminium oxide, zinc oxide or combinations thereof. More preferred whitening pigment are particles comprising titanium dioxide, zinc oxide, zirconium oxide, mica, iron oxide or a combination thereof. Even more preferred whitening pigment are particles comprising zinc oxide, zirconium oxide, titanium dioxide or a combination thereof as these materials have especially high refractive index. Still even more preferably the whitening pigment is selected from titanium dioxide, zinc oxide or a mixture thereof and most preferred whitening pigment is titanium dioxide. The average diameter of whitening pigment is typical from 15 nm to 1 micron, more preferably from 35 nm to 800 nm, even more preferably from 50 nm to 500 nm and still even more preferably from 100 to 300 nm. Amount of whitening pigment may be 0.1 to 15%, preferably 0.5 to 5% by weight of the composition.

Preferably, the composition comprises a glutamate source selected from the group consisting of glutamine, glutamine ester, glutamic acid, pyroglutamic acid, salts, and mixtures thereof. More preferably, the composition comprises pyroglutamic acid and/or salt of pyroglutamic acid. Even more preferably, the composition comprises sodium salt of pyroglutamic acid. Preferably, the glutamate source is present in amount of 0.0001 to 10% by weight of the composition, more preferably 0.001 to 6%, even more preferably 0.01 to 3% by weight of the composition.

Preferably, the composition comprises polyhydric alcohol. Polyhydric alcohols may be selected from group of glycerin, propylyene glycol, dipropylene glycol, polypropylene glycol, polyethylene glycol, sorbitol, hydroxypropyl sorbitol, hexylene glycol, 1,3-butylene glycol, isoprene glycol, ethoxylated glycerol, propoxylated glycerol or a mixture thereof. Most preferred polyhydric alcohol is glycerol known also as glycerin. The amount of polyhydric alcohol may range anywhere from 0.1 to 20%, preferably 0.5 to 15% and more preferably 2 and 10% by weight of the composition.

Preferably, the composition comprises emollient materials. Suitable emollient materials include silicones, hydrocarbons, triglycerides or a mixture thereof. These silicones may be organic, silicone-containing or fluorine-containing, volatile or non-volatile, polar or non-polar. Hydrocarbons may include mineral oil, petrolatum and polyalpha-olefins. Examples of preferred volatile hydrocarbons include polydecanes such as isododecane and isodecane (e.g. Permethyl-99A which is available from Presperse Inc.) and the C7-C8 through C12-C15 isoparaffins (such as the Isopar Series available from Exxon Chemicals). Illustrative triglycerides but not limiting are sunflower seed oil, cotton oil, canola oil, soybean oil, castor oil, borage oil, olive oil, shea butter, jojoba oil and mixtures thereof. Mono- and di-glycerides may also be useful. Particularly preferable are glyceryl monostearate and glyceryl distearate.

Preferably, the composition comprises moisturizing agents. Particularly preferred moisturizing agents includes, petrolatum, aquaporin manipulating actives, oat kernel flour, substituted urea like hydroxyethyl urea, hyaluronic acid and/or its precursor N-acetyl glucosamine, hyaluronic acid and/or its precursor N-acetyl glucosamine, or a mixture thereof.

Some compositions may include thickeners. These may be selected from cellulosics, natural gums and acrylic polymers but not limited by this thickening agent types. Among the cellulosics are sodium carboxymethyl cellulose, hydroxyethyl cellulose, hydroxypropyl methylcellulose and combinations thereof. Suitable gums include xanthan, pectin, karaya, agar, alginate gums and combinations thereof. Among the acrylic thickeners are homopolymers and copolymers of acrylic and methacrylic acids including carbomers such as Carbopol 1382, Carbopol 982, Ultrez, Aqua SF-1 and Aqua SF-2 available from the Lubrizol Corporation. Amounts of thickener may range from 0.01 to 3% by weight of the active polymer (outside of solvent or water) in the compositions.

In addition, the compositions of the invention may further include 0.5 to 10% by weight of sequestering agents, such as tetra sodium ethylenediaminetetraacetate (EDTA), EHDP or mixtures; opacifiers and pearlizers such as ethylene glycol distearate, titanium dioxide or Lytron 621 (Styrene/Acrylate copolymer); all of which are useful in enhancing the appearance or properties of the product.

The composition may comprise water in amount of 10 to 96% by weight of the composition, more preferably from 25 to 92%, even more preferably from 42 to 88%, most preferably from 55 to 82% by weight of the composition.

Preferably, the composition has a viscosity of at least 10 mPa·s, more preferably in the range 30 to 10000 mPa·s, even more preferably 50 to 5000 mPa·s, and most preferably 100 to 2000 mPa·s, when measured at 20 degrees C. at a relatively high shear rate of about 20 s⁻¹. Preferably, the composition is in the form of fluid.

Preferably, the composition is an emulsion, more preferably an oil-in-water emulsion. The skin care composition refers to a composition suitable for topical application to human skin, including leave-on and wash-off products but preferably leave-on compositions. The term “leave-on” as used with reference to compositions herein means a composition that is applied to or rubbed on the skin, and left thereon. The term “wash-off” as used with reference to compositions herein means a skin cleanser that is applied to or rubbed on the skin and rinsed off substantially immediately subsequent to application. Preferably the skin care composition means a fluid liquid, and particularly a moisturizer rather than a make-up product.

Preferably, the personal care composition is a skin care composition. The term “skin” as used herein includes the skin on the face, neck, chest, abdomen, back, arms, under arms, hands, and legs. Preferably “skin” means includes the skin on the face and under arms, more preferably skin means skin on the face other than lips and eyelids.

Preferably, the composition is a topical composition. Preferably, the composition may be in the form of cream, lotion, ointment, solution, suspension, emulsion, paste, gel, powder, powder foundation, emulsion foundation, wax foundation, or spray. More preferably, the composition may be formulated in the form of cream, lotion, ointment, emulsion, gel, or a spray.

The present invention also provides use of vitamin B3 compound and carboxymethyl cysteine compound with a weight ratio of the vitamin B3 compound to the carboxymethyl cysteine compound is at least 6:1, in the manufacture of medicants for healing and/or alleviating a skin wound; and/or regenerating skin.

Preferably the use is non-therapeutic. Preferably the method is non-therapeutic. The term non-therapeutic typically means for cosmetic purposes and not curative or therapeutic purposes.

The following examples are provided to facilitate an understanding of the invention. The examples are not intended to limit the scope of the claims.

### EXAMPLES

### Materials

| | Trade | | Abbre- | Active |
|---|---|---|---|---|
| Supplier | name | Active | viation | wt % |
| Sinerga | HAIR APP | Lysine carboxymethyl | LCC | >98% |
| | | cysteinate | | |
| Sigma-Aldrich | — | Niacinamide | — | >98% |

### Example 1

This Example demonstrates the improved wound healing effect by combining vitamin B3 compound and within a specific ratio.

The migration of epidermal keratinocytes is the basis for skin re-epithelialization during wound healing. The in vitro scratch-wound assay using monolayers of primary human epidermal keratinocytes is a straightforward and effective method to assess the migratory capacity.

Human epidermal keratinocytes (lot.: Ep21012001, Biocell) were seeded into 6-well plates, at 4.5×10⁵ cells per well and maintained in a 37° C. incubator in an atmosphere of 5% of carbon dioxide in EpiLife™ Keratinocyte Medium until the cells reaching 70 to 80% of confluence in the well. Then, the cells were cultured by fresh medium (as control) or fresh medium supplemented with different actives for 24 hours. The cells were scratched by a 5 mL of pipette tip in an identical manner to simulate the formation of wound, and images for the cells were taken immediately to record the wound. The cells were cultured again by identical fresh medium or fresh medium supplemented with different actives at 37° C. for 24 hours and images for the cells were taken again. The ratio of area before and after the further culture were obtained by using the software

Image-pro plus. The relative migration rates for difference actives were calculated by dividing the values of control and were summarized in Table 1.

TABLE 1

| | | Weight | Relative |
|---|---|---|---|
| | | ratio of | migra- |
| | | niacinamide | tion |
| | Active* | to LCC | rate |
| Control | — | — | 1 |
| A | 0.01% of Niacinamide | — | 0.94 |
| B | 0.03% of Niacinamide | — | 1.19 |
| C | 0.1% of Niacinamide | — | 1.08 |
| D | 0.01% of LCC | — | 1.04 |
| E | 0.05% of LCC | — | 1.15 |
| F | 0.01% of Niacinamide + 0.05% of LCC | 1:5 | 0.94 ^{a} |
| G | 0.01% of Niacinamide + 0.01% of LCC | 1:1 | 1.08 ^{a} |
| H | 0.03% of Niacinamide + 0.01% of LCC | 3:1 | 1.19 ^{a} |
| 1 | 0.1% of Niacinamide + 0.01% of LCC | 10:1 | 1.28 ^{b} |

*The level of actives is weight percentage based on the amount of medium
^{a} not significantly better (p < 0.05) than either of single active with at level.
^{b} significantly better (p < 0.05) than either of single active at same level.

It was evident from Table 1 that only when combining the niacinamide and lysine carboxymethyl cysteinate in a weight ratio of the present invention, the relative migration rate is significantly improved (1 vs. C and D). In contrast, when combining the niacinamide and lysine carboxymethyl cysteinate in a weight ratio not within the range of the present invention, the relative migration rate is not significantly improved (F vs. A and E; G vs. A and D; H vs. B and D).

1. A personal care or pharmaceutical composition comprising vitamin B3 compound and carboxymethyl cysteine compound, wherein the vitamin B3 compound to the carboxymethyl cysteine compound has a weight ratio of at least 6:1.

2. The personal care or pharmaceutical composition according to claim 1, wherein the carboxymethyl cysteine compound comprises carboxymethyl cysteine, an ester of carboxymethyl cysteine, and/or a salt of carboxymethyl cysteine.

3. The personal care or pharmaceutical composition according to claim 2, wherein the carboxymethyl cysteine compound comprises a salt of carboxymethyl cysteine and preferably the carboxymethyl.

4. The personal care or pharmaceutical composition according to claim 1, wherein the carboxymethyl cysteine compound is present in an amount of at least 0.00001% and no greater than 10% by weight of the composition.

5. The personal care or pharmaceutical composition according to claim 1, wherein the vitamin B3 compound comprises niacin, niacinamide, or a mixture thereof.

6. The personal care or pharmaceutical composition according to claim 1, wherein the vitamin B3 compound is present in an amount of at least 0.00001% by weight of the composition and no more than 10% by weight of the composition.

7. The composition according to any one of the preceding claims The personal care or pharmaceutical composition according to claim 1, wherein the vitamin B3 compound to the carboxymethyl cysteine compound has a weight ratio of 6:1 to 10000:1.

8. The composition according to any one of the preceding claims 3 to 7 The personal care or pharmaceutical composition according to claim 1, wherein the vitamin B3 compound to the lysine carboxymethyl cysteinate has a weight ratio of 6:1 to 10000:1.

9. The personal care or pharmaceutical composition according to claim 1, wherein the composition is an oil-in-water emulsion.

10. The personal care or pharmaceutical composition according to claim 1, wherein the composition comprises water in an amount of 10 to 96% by weight of the composition.

11. (canceled)

12. A method of healing and/or alleviating a skin wound; and/or regenerating skin comprising a step of topically applying to the skin the personal care or pharmaceutical composition according to claim 1.

13. (canceled)

14. The personal care or pharmaceutical composition according to claim 2, wherein the carboxymethyl cysteine compound comprises lysine carboxymethyl cysteinate.

15. The personal care or pharmaceutical composition according to claim 1, wherein the carboxymethyl cysteine compound is present in an amount of at least 0.01% by weight of the composition and no greater than 3% by weight of the composition.

16. The personal care or pharmaceutical composition according to claim 1, wherein the vitamin B3 compound comprises niacinamide.

17. The personal care or pharmaceutical composition according to claim 1, wherein the vitamin B3 compound is present in an amount of at least 0.001% by weight of the composition and no more than 5% by weight of the composition.

18. The personal care or pharmaceutical composition according to claim 1, wherein the vitamin B3 compound to the carboxymethyl cysteine compound has a weight ratio of 6:1 to 100:1.

19. The personal care or pharmaceutical composition according to claim 1, wherein the vitamin B3 compound to the carboxymethyl cysteine compound has a weight ratio of 7:1 to 50:1.

20. The personal care or pharmaceutical composition according to claim 1, wherein the vitamin B3 compound to the carboxymethyl cysteine compound has a weight ratio of 8:1 to 20:1.

21. The personal care or pharmaceutical composition according to claim 1, wherein the vitamin B3 compound to the lysine carboxymethyl cysteinate has a weight ratio of 7:1 to 50:1.

22. The personal care or pharmaceutical composition according to claim 1, wherein the composition comprises water in an amount of 42 to 88% by weight of the composition.
